(19) United States
(12) Patent Application Publication
(10) Pub. No.: US 2024/0105328 A1
(43) Pub. Date: Mar. 28, 2024

(54) NEURAL NETWORK SIMULATOR FOR ULTRASOUND IMAGES AND CLIPS

(71) Applicant: Ultrasight Ltd., Nes Ziona (IL)

(72) Inventors: Jonathan SHLOMI, Givat Ada (IL); Itay KEZURER, Rehovot (IL); Yaron LIPMAN, Kibbutz Netzer Sereni (IL)

(21) Appl. No.: 18/469,641

(22) Filed: Sep. 19, 2023

Related U.S. Application Data
(60) Provisional application No. 63/376,108, filed on Sep. 19, 2022.

(51) Int. Cl. G16H 40/63; A61B 8/00; G06N 3/0475; G06N 3/08; G06T 15/08; G09B 23/28

(52) U.S. Cl. CPC G16H 40/63; A61B 8/4254; G06N 3/0475; G06N 3/08; G06T 15/08; G09B 23/286; G06T 2210/41

(57) ABSTRACT

An ultrasound image simulator includes a generative neural network to receive ultrasound probe position and orientation and to generate at least one simulated ultrasound image or clip of a body part of a subject. The generative neural network is trained on a multiplicity of 2D ultrasound images or clips of said body part taken from a plurality of ultrasound probe positions and orientations.

### CROSS REFERENCE TO RELATED APPLICATIONS

This application claims priority from U.S. provisional patent application 63/376,108, filed Sep. 19, 2022, which is incorporated herein by reference.

### FIELD OF THE INVENTION

The present invention relates to simulators generally and to neural network simulators for ultrasound images and clips in particular.

### BACKGROUND OF THE INVENTION

Generating synthetic ultrasound images is difficult and typically requires complicated modeling of the 3D structure (shape, density) of the body using 3D MRI or ultrasound data of the internal structure.

As shown in FIG. 1, to which reference is now made, existing simulation methods for producing images have three components: an ultrasound beam generator 10 which produces a simulated sound beam, a modeler 12 of the interaction between the ultrasound beam and a 3D model of the internal organs of the patient, and an ultrasound image generator 14 which forms ultrasound images based on the sound waves returning from the internal organs. The 3D model is typically generated by a 3D modeler 16 from 3D type data, such as MRI scans and/or 3D ultrasound scans and/or artificial 3D models created using 3D modelling software.

The simulations may utilize linear methods, ray tracing or numerical solutions of wave equations.

The article by J. Jensen and N. Svendsen, “Calculation of pressure fields from arbitrarily shaped, apodized, and excited ultrasound transducers,” IEEE Transactions on Ultrasonics, Ferroelectrics, and Frequency Control, vol. 39, no. 2, pp. 262-267, 1992, describes an exemplary linear method. In this method, the sound beam profile is simulated by integrating spherical waves from each point in the ultrasound transducer surface. The response from matter is computed as a convolution of the beam profile and the material scattering density. This represents an assumption that each point in the material scatters the sound wave from the beam weakly and independently of the other points. Unfortunately, this method does not simulate ultrasound effects, such as shadowing or refraction.

The articles B. Burger, S. Bettinghausen, M. Radle, and J. Hesser, “Real-time gpu-based ultrasound simulation using deformable mesh models,” IEEE transactions on medical imaging, vol. 32, no. 3, pp. 609-618, 2012; and O. Mattausch and O. Goksel, “Monte-carlo ray-tracing for realistic interactive ultrasound simulation,” in Proceedings of the Eurographics Workshop on Visual Computing for Biology and Medicine, 2016, pp. 173-181, describe ray tracing methods.

These methods assume the ultrasound sound beam can be modeled as a ray. They simulate the refraction and reflection of the sound wave as it passes through the material, but ignore the wave-like nature of the sound beam. A 3D mesh model is used to describe the anatomy of the organ whose image is being generated, and the different propagation properties of different materials are provided as parameters to the simulator. The images are post processed to generate the speckle pattern of ultrasound waves.

The article E. S. Wise, B. T. Cox, J. Jaros, and B. E. Treeby, “Representing arbitrary acoustic source and sensor distributions in Fourier collocation methods,” The Journal of the Acoustical Society of America, vol. 146, no. 1, p. 278, July 2019, issn: 1520-8524, describes a method utilizing numerical solutions of wave equations.

This method discretizes space and time, and models the propagation of the sound wave from the transducer as well as its interaction with the internal organs. The model is generated by solving differential equations on a grid. They are very accurate but time-consuming. They require a voxel map of the internal organs, and parameters for describing the density of the internal organs.

Other methods use a neural network to add ultrasound effects to a simulated 2D slice as generated from the systems described above. These 2D slices are used as input to a neural network which generates the ultrasound image.

This is described in the articles by F. Tom and D. Sheet, simulating patho-realistic ultrasound images using deep generative networks with adversarial learning, 2017, and by B. Peng, X. Huang, S. Wang, and J. Jiang, “A real-time medical ultrasound simulator based on a generative adversarial network model,” in 2019 IEEE International Conference on Image Processing (ICIP), 2019, pp. 4629-4633. The neural network is responsible for simulating the ultrasound effects, but not for the geometry of the image.

All of these methods require some sort of 3 dimensional modeling provided through either a 3D (volumetric) imaging system (e.g., 3D ultrasound, MRI, CT scans) or artificial parametric 3D models.

### SUMMARY OF THE PRESENT INVENTION

There is therefore provided, in accordance with a preferred embodiment of the present invention, an ultrasound image simulator including a generative neural network to receive ultrasound probe position and orientation and to generate at least one simulated ultrasound image or clip of a body part of a subject. The generative neural network is trained on a multiplicity of 2D ultrasound images or clips of said body part taken from a plurality of ultrasound probe positions and orientations.

Moreover, in accordance with a preferred embodiment of the present invention, the multiplicity of 2D ultrasound images or clips and the plurality of ultrasound probe positions and orientations are generated by an ultrasound guidance system. Alternatively, they are generated by a probe motion sensing system.

Further, in accordance with a preferred embodiment of the present invention, the images or clips are associated with a multiplicity of parameters. For example, the parameters might be parameters of an ultrasound probe, patient parameters, operator parameters or noise parameters.

Still further, in accordance with a preferred embodiment of the present invention, the at least one simulated ultrasound image or clip has an artificial or non-human element therein.

Moreover, in accordance with a preferred embodiment of the present invention, the generative neural network also includes a latent variable provider.

Further, in accordance with a preferred embodiment of the present invention, the generative neural network is trained with a latent variable neural network receiving the multiplicity of 2D ultrasound images or clips and the plurality of ultrasound probe positions and orientations.

Moreover, in accordance with a preferred embodiment of the present invention, the generative neural network includes a diffusion model neural network.

Alternatively, in accordance with a preferred embodiment of the present invention, the generative neural network includes a volumetric neural network, a slicer and a rendering neural network. The volumetric neural network receives the ultrasound probe position and orientation and generates volume data of a body part for the ultrasound probe position and orientation. The slicer extracts a slice of the volume associated with the position and orientation of the ultrasound probe. The rendering neural network renders the slice as a simulated ultrasound image of the body part.

Further, in accordance with a preferred embodiment of the present invention, both the volumetric neural network and the rendering neural network are trained with an empirical risk minimization training procedure and utilize the same loss function.

Moreover, in accordance with a preferred embodiment of the present invention, the simulator may be used in a system producing synthetic data for training an ultrasound based machine learning unit.

Further, in accordance with a preferred embodiment of the present invention, the ultrasound based machine learning unit may be a classifier, a segmenter or a regressor.

Still further, in accordance with a preferred embodiment of the present invention, the ultrasound based machine learning unit includes a navigation neural network under training and generates probe movement instructions for an ultrasound probe on a virtual subject, and a probe position updater to convert the movement instructions to position and orientation of the ultrasound probe.

Moreover, in accordance with a preferred embodiment of the present invention, the simulator may be used in a system producing synthetic data for training a person to perform ultrasound scans of an artificial body with an ultrasound probe, together with sonographer training software to receive the at least one simulated ultrasound image or clip and to provide instructions to the person.

Moreover, in accordance with a preferred embodiment of the present invention, the simulator may be used in a system to improve a partial or corrupted image of a body part. The generative neural network generates a simulated ultrasound image of the body part and the system includes a comparator to determine a difference between the simulated ultrasound image with the partial or corrupted image and an optimizer to update the multiplicity of parameters and/or the ultrasound probe position and orientation to reduce the difference, thereby to produce an improved version of the partial or corrupted image of the body part.

Further, in accordance with a preferred embodiment of the present invention, the system can be used for image completion and/or image correction and/or image noise reduction and/or 3D reconstruction and/or new view synthesis.

There is also provided, in accordance with a preferred embodiment of the present invention, a method for generating an ultrasound image. The method includes generating at least one simulated ultrasound image or clip of a body part of a subject with a generative neural network in response to ultrasound probe position and orientation. The generative neural network is trained on a multiplicity of 2D ultrasound images or clips of the body part taken from a plurality of ultrasound probe positions and orientations.

Further, in accordance with a preferred embodiment of the present invention, the method includes generating the multiplicity of 2D ultrasound images or clips and the plurality of ultrasound probe positions and orientations by an ultrasound guidance system.

Still further, in accordance with a preferred embodiment of the present invention, the method includes generating the plurality of ultrasound probe positions and orientations by a probe motion sensing system.

Moreover, in accordance with a preferred embodiment of the present invention, the method also includes providing latent variables to the generative neural network.

Further, in accordance with a preferred embodiment of the present invention, the method includes training the generative neural network with a latent variable neural network receiving the multiplicity of 2D ultrasound images or clips and the plurality of ultrasound probe positions and orientations.

Still further, in accordance with a preferred embodiment of the present invention, the generative neural network includes a diffusion model neural network.

Moreover, in accordance with a preferred embodiment of the present invention, the generating includes generating volume data of a body part for the ultrasound probe position and orientation with a volumetric neural network in response to the ultrasound probe position and orientation, extracting a slice of the volume associated with the position and orientation of the ultrasound probe, and rendering the slice as a simulated ultrasound image of the body part with a rendering neural network.

Further, in accordance with a preferred embodiment of the present invention, the method includes training both the volumetric neural network and the rendering neural network with an empirical risk minimization training procedure that utilizes the same loss function.

There is also provided, in accordance with a preferred embodiment of the present invention, a method for producing synthetic data for training an ultrasound based machine learning unit. The method includes receiving at least one of the multiplicity of parameters, and generating at least one the simulated ultrasound image or clip for the at least one parameter with the generative neural network.

Moreover, in accordance with a preferred embodiment of the present invention, the ultrasound based machine learning unit is one of: a classifier, a segmenter and a regressor. Alternatively, the ultrasound based machine learning unit includes a navigation neural network under training which generates probe movement instructions for an ultrasound probe on a virtual subject and the method includes converting the movement instructions to position and orientation of the ultrasound probe.

There is also provided, in accordance with a preferred embodiment of the present invention, a method for producing synthetic data for training a person to perform ultrasound scans of an artificial body with an ultrasound probe. The method includes receiving at least one of the multiplicity of parameters, generating at least one the simulated ultrasound image or clip for the at least one parameter with the generative neural network and a sonographer training software providing instructions to the person.

Finally, there is also provided, in accordance with a preferred embodiment of the present invention, a method to improve a partial or corrupted image of a body part, The method includes generating at least one the simulated ultrasound image or clip with the generative neural network, determining a difference between the simulated ultrasound image with the partial or corrupted image, and updating the multiplicity of parameters and/or the ultrasound probe position and orientation to reduce the difference, thereby to produce an improved version of the partial or corrupted image of the body part.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram illustration of prior art simulation methods;

FIG. 2 is a block diagram illustration of a novel simulator for ultrasound images and clips, constructed and operative in accordance with a preferred embodiment of the present invention;

FIG. 3 is a schematic illustration of a sliced heart showing an exemplary ultrasound view, useful in understanding the simulator of FIG. 2;

FIG. 4 is a schematic illustration of a training dataset for the simulator of FIG. 2;

FIGS. 5A and 5B are block diagram illustrations of an embodiment of the simulator of FIG. 2 and its training elements, respectively, constructed and operative in accordance with a preferred embodiment of the present invention;

FIG. 6 is a graphic illustration of exemplary simulated images generated by the simulator of FIG. 2;

FIGS. 7A and 7B are block diagram illustrations of a latent variable, alternative embodiment of the simulator of FIG. 5A and its training elements, respectively, constructed and operative in accordance with a preferred embodiment of the present invention;

FIGS. 8A and 8B are block diagram illustrations of a further alternative embodiment of the simulator of FIG. 2 using a direct generative neural network without and with a latent variable model, respectively, constructed and operative in accordance with a preferred embodiment of the present invention;

FIG. 8C is a block diagram illustration of a training portion of the direct generative neural network of FIGS. 8A and 8B;

FIG. 8D is a block diagram illustration of the direct generative neural network of FIGS. 8A and 8B during operation;

FIG. 9 is a block diagram illustration of a system producing synthetic data for machine learning systems using the simulator of FIG. 2;

FIG. 10 is a block diagram illustration of a training aid for sonographers using the simulator of FIG. 2; and

FIG. 11 is a block diagram illustration of an image improvement system using the simulator of FIG. 2.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that there are thousands of 2D ultrasound clips available, such as from doctor's offices, hospitals, etc. These are typically of standard views of internal organs. For example, the standard views of the heart are a 2 chamber view, 4 chamber view, and 5 chamber view. Associated with some of these ultrasound clips are patient history and/or diagnoses. This significant amount of data may be simple and relatively inexpensive to collect compared to the costs of collecting 3D data.

Applicant has further realized that U.S. Pat. No. 11,593,638 to New York University and Yeda Research and Development Co. Ltd., incorporated herein by reference, which provides changes in probe position and orientation to guide a user towards standard ultrasound views, may provide a database of 2D ultrasound images taken from a plurality of ultrasound probe positions and orientations. Applicant has realized that the data from U.S. Pat. No. 11,593,638 may provide a relatively simple source of probe data.

Applicant has further realized that the easily collected 2D ultrasound images may be utilized directly in a generative neural network to aid in generating synthetic ultrasound data, particularly of unusual pathologies, and that the synthetic ultrasound clips may be utilized for training sonographers. Furthermore, the easily collected 2D ultrasound images may also include non-tissue elements, such as pacemaker, replacement valves, etc., which are difficult to obtain in a 3D machine such as an MRI or a CT.

Applicant has realized that a generative neural network may simulate the physics both of ultrasound beam formation and of ultrasound wave propagation in biological tissue. This may provide faster image generation times (for use in real-time systems) and it may greatly simplify the development process, thereby allowing the simulation of many different types of ultrasound exams.

Furthermore, Applicant has realized that the prior methods are limited to simulating ultrasound clips only for the specific people included in the 3D data. For example, if in the available dataset there is a person A data with some BMI value and some cardiac condition unique to person A, there is no way to generate ultrasound clips of a person with the cardiac condition and a different BMI value.

Applicant has realized that generative neural networks may generate novel samples that are similar but not identical to the samples in the training data. Continuing the example above, if there are people with a range of BMI values in the dataset, and only some of them have a specific condition, the network will be able to generate samples with that condition for the full BMI range.

Moreover, due to the present invention's use of 2D ultrasound data rather than 3D data, the variety of patients and situations is much broader than that of the prior art. The generative neural network may thus model the shape space of human organs and may generate ultrasound images of a variety of shapes and conditions, without having to collect the data from a specific human having a specific set of parameters and/or diagnosis.

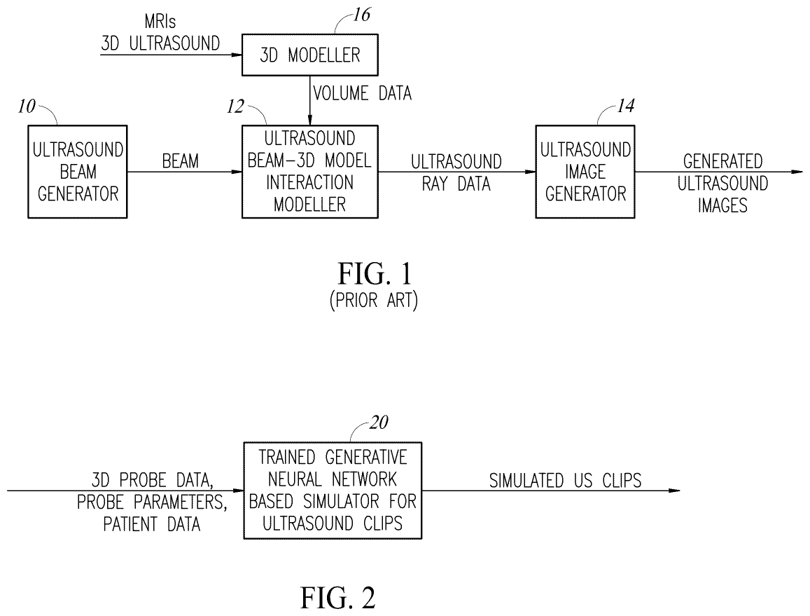

Reference is now made to FIG. 2, which illustrates a simulator 20 for ultrasound images and clips which generates ultrasound clips from input probe position and orientation data related to a particular 2D view, or slice, of that organ, as well as probe parameters and patient data, such as the patient's BMI, the patient's age, any diagnosed disease that the patient may have, etc. Other parameters that may be included may be operator parameters and noise parameters.

In accordance with a preferred embodiment of the present invention, simulator 20 may be implemented as a generative neural network-based simulator, trained with a 13ultiplicity of 2D ultrasound images of at least one body part or organ, taken at associated probe positions and orientations, as well as probe parameters and patient data.

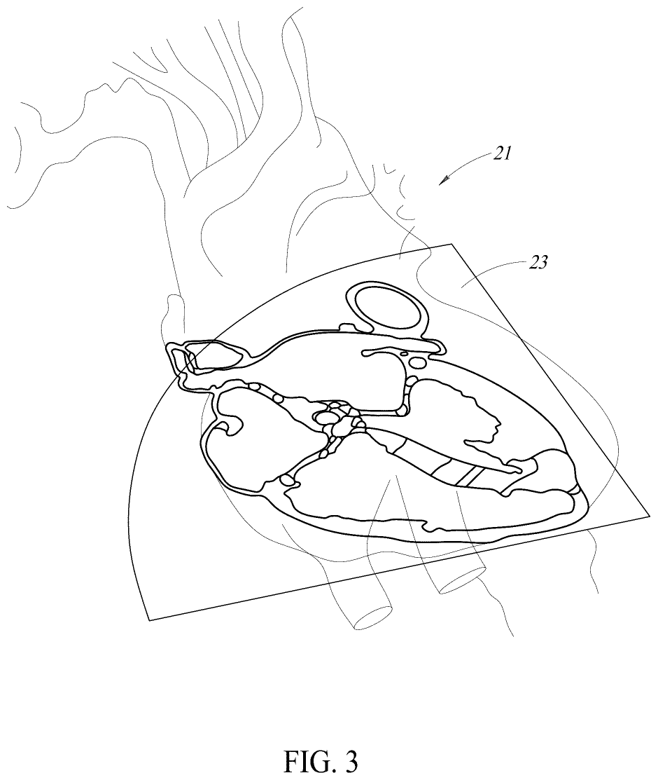

Applicant has realized that each ultrasound image in a training dataset may be mapped to a slice in 3D space at a point in time. This is shown in FIG. 3, to which reference is now made. FIG. 3 shows a heart 21 with an ultrasound view 23 sliced through it. View 23 shows a standard 4 chamber view of heart 21.

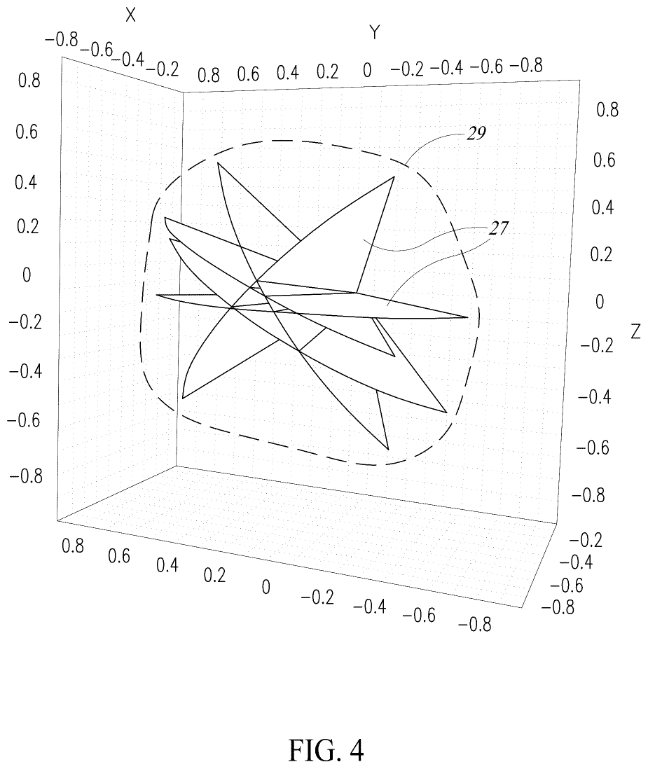

Thus, the training data represents a plurality of different views of the same 4D space (three spatial dimensions and one time dimension) or organ. FIG. 4, to which reference is now briefly made, shows an exemplary training dataset of multiple views 27 slicing through the same organ 29.

Applicant has further realized that a generative neural network may be trained on such a training dataset and may generate from these a 3D model of organ 29.

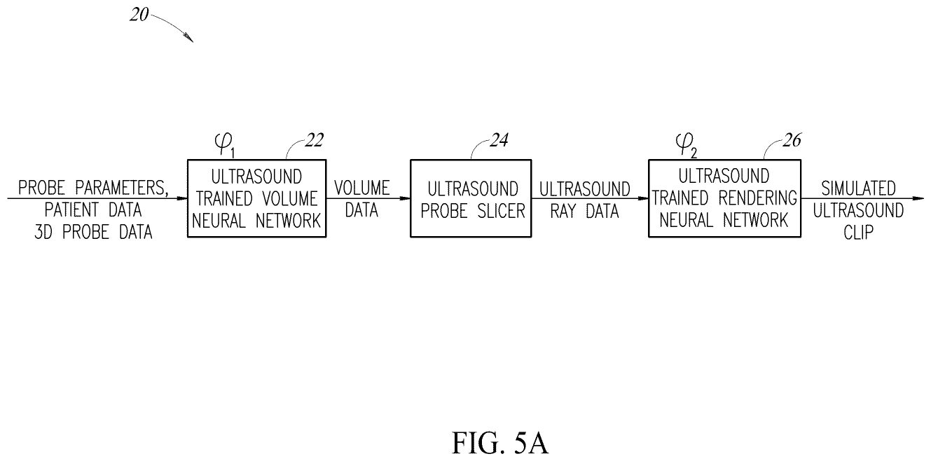

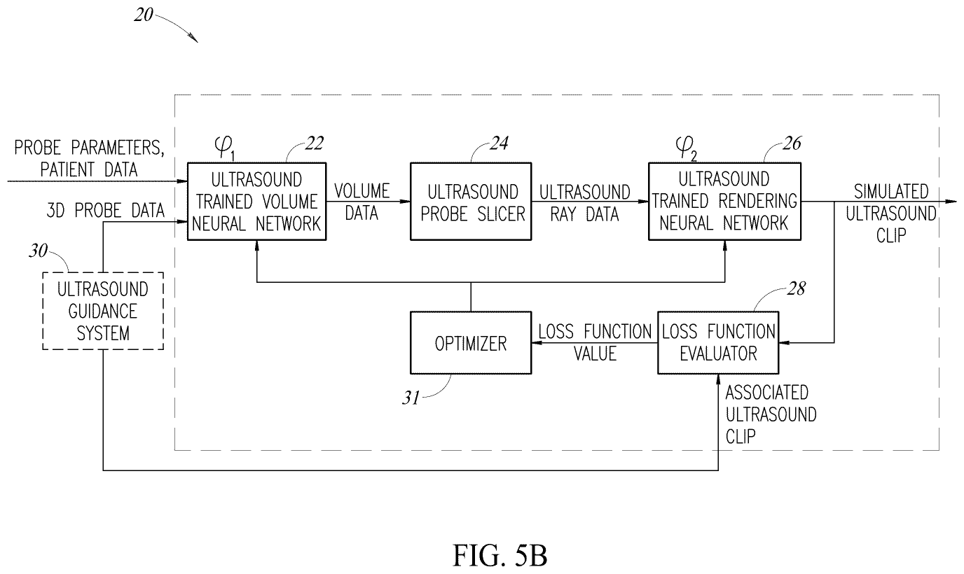

Reference is now made to FIG. 5A, which shows one embodiment of simulator 20 of FIG. 2, and to FIG. 5B which shows simulator 20 in its training process. Simulator 20 may comprise an ultrasound trained, volumetric neural network 22, to generate an approximate 3D model of organ 29, an ultrasound probe slicer 24 to “slice” through the 3D model along a viewing line of an ultrasound transducer to generate views 27 and an ultrasound trained, rendering neural network 26 to render ultrasound images for each of views 27, with all the effects that occur in an ultrasound image.

Since generating the 3D model is difficult to do analytically, ultrasound trained volumetric neural network 22 may act as a non-linear, volumetric function ϕ1(p, y, z) to generate the 3D model, where p is a position in 4D space-time, y is an input parameter, and z is an optional variable for latent variables, as discussed in more detail hereinbelow. Input parameters may be parameters of the ultrasound transducer, patient data, which organ is viewed and its properties, relative time/sequential data, pathology (if present), and any other physical properties. Exemplary parameters of the transducer may be position and orientation of the probe relative to the organ, shape of the ultrasound field of view, machine/transducer type, etc. Exemplary patient data may be gender, age, BMI, and special conditions. Exemplary operator parameters may be how steady the operator's hand is. In addition, there may be noise parameters defining how clear the ultrasound image is.

Ultrasound trained, volumetric neural network 22 may be implemented as an MLP (multi-layer perceptron neural network) or as learnable parameters on a voxel grid in 4D space-time.

As is known in the art, neural networks of any architecture comprise a large number of numerical parameters known as “weights”, “learnable parameters” or simply “network parameters”, which are denoted by θ. The output of a given neural network architecture to a specific input depends on the specific values of θ. When a neural network is first initialized, its learnable parameters are random, and it does not provide the desired output. During training, these learnable parameters are determined.

Every neural network must go through an optimization process (commonly referred to as a “training process”) in which its learnable parameters are adjusted using a loss function which quantifies the difference between the output of the network (which is initially random) and the desired output as provided by the training data. In the optimization process, the actual output of a network and the desired output are compared using the loss function, which gives a numerical value to their difference. The derivative of the loss function with respect to each network learnable parameter is then computed numerically. The derivative indicates how the value of the loss function will change if that specific learnable parameter will change. The learnable parameter is then changed in a way which decreases the loss function. Many repetitions of this step for all examples in the training data lead to a neural network which provides the desired output for a given input.

Ultrasound probe slicer 24 may define the 2D view of the probe towards organ 29 and thus, may select the points of the 3D model of organ 29 that form the ultrasound image field of view. Ultrasound probe slicer 24 may provide the selected points pi to rendering neural network 26.

Given a probe position and orientation, ultrasound probe slicer 24 is tasked with determining which points in 3D space are relevant for the formulation of the ultrasound image. This is performed using a form of ray casting—a computer graphics technique which simulators the propagation of light towards a camera, in order to render 2D images from a 3D scene. However, ultrasound probe slicer 24 sends rays through the 3D volume from the probe position in a direction determined by the probe orientation and in a pattern that matches the transmission of ultrasound waves, and selects points along these rays to represent the pixels of the 2D ultrasound image.

Ultrasound trained, rendering neural network 26 may be implemented as a transformer neural network and may operate either locally (on a single point pᵢ) or on groups of points at a time, using models such as transformers, which are described in the article by A. Vaswani, N. Shazeer, N. Parmar, et al., “Attention is All you Need,” in Advances in Neural Information Processing Systems, I. Guyon, U. V. Luxburg, S. Bengio, et al., Eds., vol. 30, Curran Associates, Inc., 2017. Models that operate on sets of points can model the directional dependence of the image formation, for ultrasound effects such as shadowing and refraction, as described in the article by M. Demi, “The basics of ultrasound,” in Comprehensive Biomedical Physics, A. Brahme, Ed., Oxford: Elsevier, 2014, pp. 297-322, isbn: 978-0-444-53633-4.

In order to train simulator 20, the probe data, patient data, and any other parameters, and the ultrasound image or clip associated with that data may be provided from any source. For example, the probe position and orientation data may be provided from a probe motion sensing system, such as the 3D Guidance trakSTAR, commercially available from Northern Digital Inc., of Canada, and the ultrasound images or clips may be provided from an ultrasound machine.

FIG. 5B shows an alternative embodiment where the ultrasound images or clips and the probe position and orientation which produced them may be provided by an ultrasound guidance system 30, such as that described in U.S. Pat. No. 11,593,638. Ultrasound guidance system 30, which is neural network based, may guide a user, who may be a trained sonographer or an untrained user, to move an ultrasound probe to a desired position and orientation in order to view a standard view of organ 29. In accordance with a preferred embodiment of the present invention, ultrasound guidance system 30 may generate probe position and orientation information associated with the ultrasound images or clips, at the standard views and/or during motion from a current probe position and orientation towards the desired position and orientation.

For training, ultrasound guidance system 30 may provide probe position and orientation information as input to volumetric neural network 22 and may provide the associated ultrasound images or clips to a loss function evaluator 28. Volumetric neural network 22 may also receive the relevant patient data and probe parameters.

As can be seen from FIG. 5B, for simulator 20, an optimizer 31 may update both neural networks 22 and 26 with the derivative of the loss function value produced by loss function evaluator 28. Applicant has realized that taking the derivative of the loss value with respect to the learnable parameters of both rendering neural network 26 and volumetric neural network 22 is possible since the generated ultrasound images are influenced by both networks. Using the derivative of the loss function with respect to the learnable parameters allows changing those learnable parameters in order to make the generated ultrasound images match the desired output.

The training data may be composed of pairs of input and desired output, where the input may be the input probe information and patient data and the desired output may be the associated ultrasound images.

Loss function evaluator 28 may receive the associated ultrasound images or clips from ultrasound guidance system 30 as well as the simulated ultrasound images or clips which are the output of rendering neural network 26, and may generate a value for the loss function. In one embodiment of the present invention, volumetric neural network 22 and rendering neural network 26 may be trained using an empirical risk minimization (ERM) training procedure of the form:

$$\\ \begin{matrix}L ( θ ) = \frac{1}{m} {\sum }_{i = 1}^{m} ℓ ( {x}_{i} , ( Φ ( {y}_{i} , {z}_{i} ) ) & equation 1\end{matrix}$$

where [symbol] is a loss function, xᵢ is the input to simulator 20, Φ(yᵢ, zᵢ) is the output of simulator 20, and m is the number of examples used in a batch of training.

The loss function may be written [symbol] (x, x′) and one exemplary loss function, which measures the difference between associated ultrasound image x and simulated ultrasound image x′, may be:

[symbol]

(x,x′)=Σ_{I}|Iₓ−I_{x′}| equation 2

where Iₓ and Iₓ, are the intensities of the I-th pixel in the images x and x′, respectively.

After many iterations, simulator 20 may be trained. At this point, probe data, from guidance system 30 or from some other source of probe data, may be provided to simulator 20 to produce simulated ultrasound images or clips.

An exemplary simulator 20 was trained to generate cardiac ultrasound images, using training data from a single patient. The training data consisted of four clips, each with 32 frames, of the following canonical cardiac views: Apical four chamber view (denoted by 4C), two chambers view (denoted by 2C), five chambers view (denoted by 5C) and three chambers view (denoted by 3C). The clips were annotated with the probe orientation and position relative to the 4C view, which is defined as the reference for the coordinate system. The clips were also annotated with their timestamp within the cardiac cycle, normalized to an interval of (0, 1).

Volumetric neural network 22, which implements function ϕ1, was implemented as an MLP with 5 layers of 512 neurons. The input to it was the position of a point of a probe in space-time, and its output was a 512 dimensional vector. Rendering neural network 26, which implements function ϕ₂, was implemented as a convolutional neural network (CNN). After selection by ultrasound probe slicer 24, the 512 dimensional vectors from each selected point were provided to rendering neural network 26 to produce the final image.

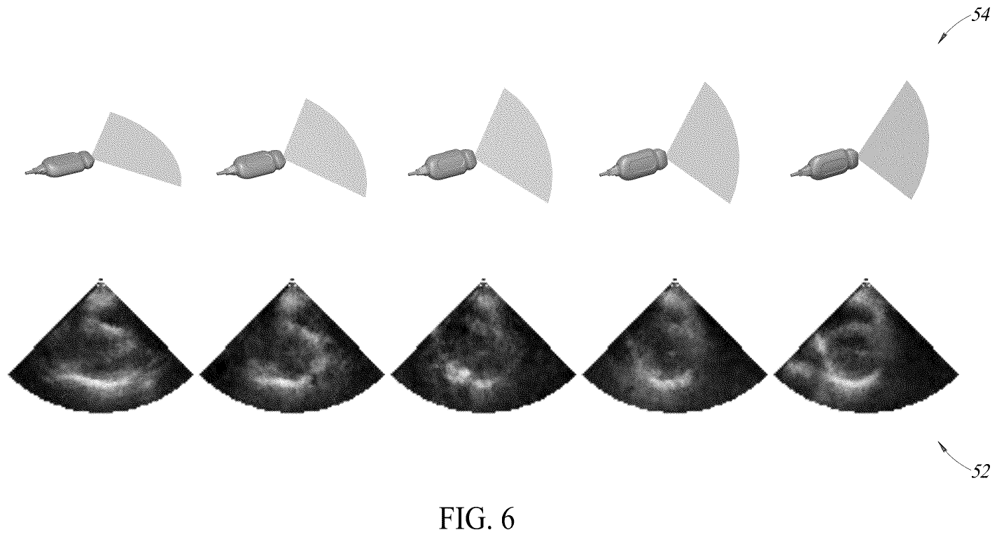

Row 52 of FIG. 6 shows a synthetic sequence generated by simulator 20. Row 54 shows the orientation of the ultrasound field of view for each frame. It will be appreciated that the images of the synthetic sequence are very realistic ultrasound images.

Although not shown in FIG. 6, it will be appreciated that simulator 20 may generate ultrasound images for ‘in-between’ probe positions not found in the original ultrasound clips. This may be particularly useful for viewing various diseased body parts for which there are only a few original ultrasound clips for training and these are typically only of the standard views. In these situations, simulator 20 may generate the non-standard views of the diseased body part.

Similarly, simulator 20 may generate various body parts of people of various BMIs, even if the training data has only sparse ultrasound clips from people with the extreme BMIs. Thus, simulator 20 may generate synthetic ultrasound clips for a variety of physiques and may thus model the shape space of human organs. As a result, simulator 20 may generate synthetic ultrasound images for a variety of shapes and conditions.

Applicant has further realized that, if simulator 20 is provided with a few training images or clips of a body part with an artificial or non-human element therein (e.g., a pacemaker in a heart, a pin in a bone, etc.), simulator 20 may generate ultrasound images of that body part with that artificial element therein for other patients, including those with other parameters.

As described in more detail hereinbelow, simulator 20 may greatly simplify the process of developing ultrasound methods and machines, as simulator 20 may easily simulate many different types of ultrasound exams, for patients with many different parameters, typically at sufficiently fast speed to be useful in real-time systems.

Applicant has realized that simulator 20 may generate clips which may provide an amalgamation (sum, or average) of the input clips rather than simulated clips which are more particular for a particular patient parameter (such as BMI). For example, clips of fat people may be amalgamated with clips of thin people.

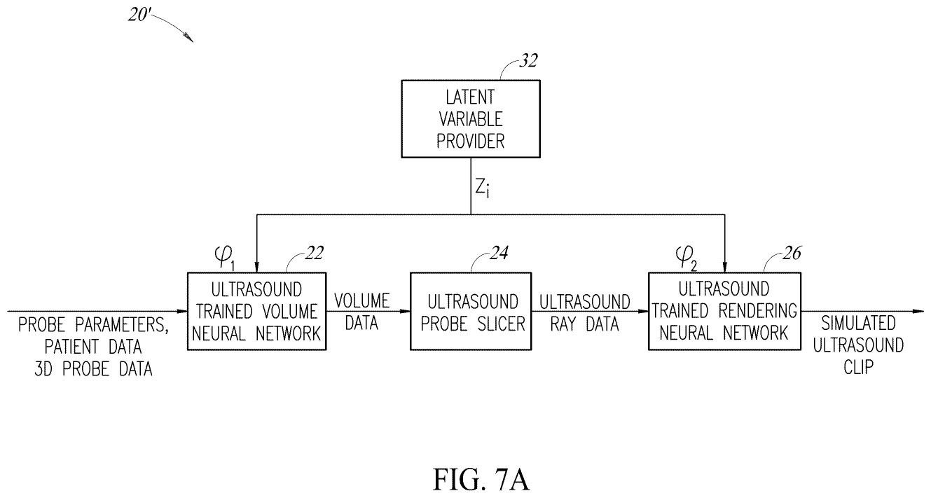

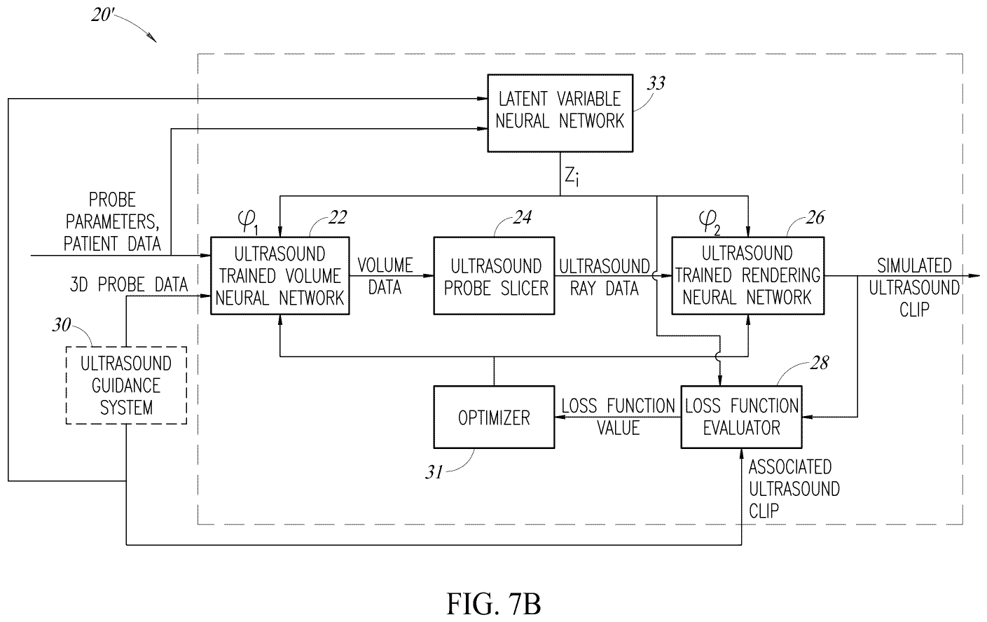

In an alternative embodiment, shown in FIGS. 7A and 7B to which reference is now made, the simulator, here labeled 20′, may also comprise a latent variable model provider 32 for operation and a latent variable model neural network 33 for training, both of which may add a distribution for particular parameters. This may ensure that the simulated ultrasound images are more closely similar to real images from patients with a selected parameter, rather than receiving an average of all patients having the selected parameter.

To do this, during training, latent variable model neural network 33 may add a random variable Z, with a normal distribution to the input of neural networks 22 and 26. Random variable Z may have a plurality of elements each of which may handle the uncertainty in one of a number of parameters, such as the actual shape of organ 29, movement of the organ inside the patient body, uncertainty in the position of the transducer, uncertainty in the time, and the variety of physiques of the patients.

During training (FIG. 7B), latent variable model neural network 33 may receive the 3D probe data, such as from guidance system 30 (FIG. 5B), the patient data, and the associated ultrasound clip and may assign a numerical value zᵢ, for example, between 0 and 1, to each pair of 3D probe data yᵢ and ultrasound image or clip xᵢ, such that the distribution of the random variable has a normal distribution (e.g. with a mean of 0 and a standard deviation of 1). For example, common cases may be at the center of the distribution while rare cases may be at the tail of the distribution. Latent variable model neural network 33 may assign random variables zᵢ in such a way that similar (xᵢ, yᵢ) pairs may have similar random variables zᵢ.

Latent variable model neural network 32 may be implemented using a standard image to feature vector model, such as are described in the articles by J. Ho, C. Saharia, W. Chan, D. J. Fleet, M. Norouzi, and T. Salimans, “Cascaded diffusion models for high fidelity image generation.,” J. Mach. Learn. Res., vol. 23, pp. 47-1, 2022, and R. T. Q. Chen, Y. Rubanova, J. Bettencourt, and D. K. Duvenaud, “Neural Ordinary Differential Equations,” in Advances in Neural Information Processing Systems, vol. 31, Curran Associates, Inc., 2018. Alternatively, latent variable model neural network 32 may be separately trained as part of a variational autoencoder (VAE), such as described in the article by D. P. Kingma and M. Welling, “An introduction to variational autoencoders,” Foundations and Trends® in Machine Learning, vol. 12, no. 4, pp. 307-392, 2019.

In FIG. 7B, latent variable model neural network 33 is trained as part of simulator 20′ and, for this embodiment, the loss function [symbol] (x, x′) in equation 2 is augmented with additional terms. The additional loss terms ensure both that the probability distribution of zi is simple/regularized (e.g., normal or another desired distribution) and that the likelihood of the training data is maximal given the trained generator and encoder. This standard procedure for training latent variable models.

An example of these additional loss terms is the KL divergence loss. If latent variable model neural network 33 assigns to each entry in the training data a random value zᵢ value using the same procedure as a variational auto encoder, then each entry in the training dataset will have an associated mean μ and standard deviation a value, which the latent variable model neural network 33 used to generate the zᵢ value. The per-random variable parameters, and a μᵢ are entered into a KL loss term, as follows:

L(θ)=−½Σᵢ₌₁^{m}(1+log(σᵢ²)−μᵢ²−σᵢ²) equation 3

where the sum is over the different training samples. This loss term is minimized when the mean (over the training dataset) of the μ values is 0 and the mean of the σ values is 1. This loss term, combined with reconstruction loss described above, ensures that both the samples are correctly reconstructed and that each entry is assigned a zᵢ value such that they are normally distribued.

After training, when simulator 20′ is operative (FIG. 7A), latent variable model neural network 33 is replaced with latent variable provider 32 which may provide to simulator 20′ randomly sampled values for z according to its distribution.

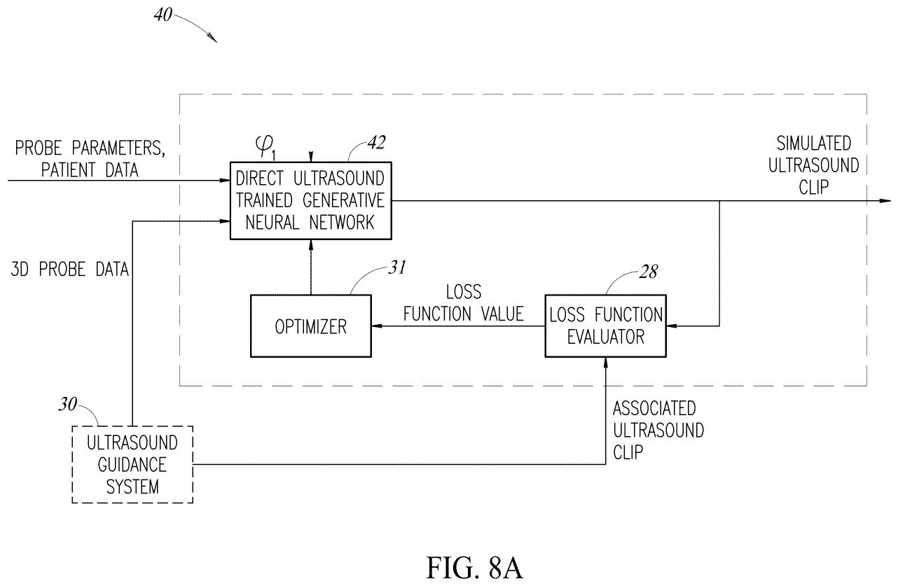

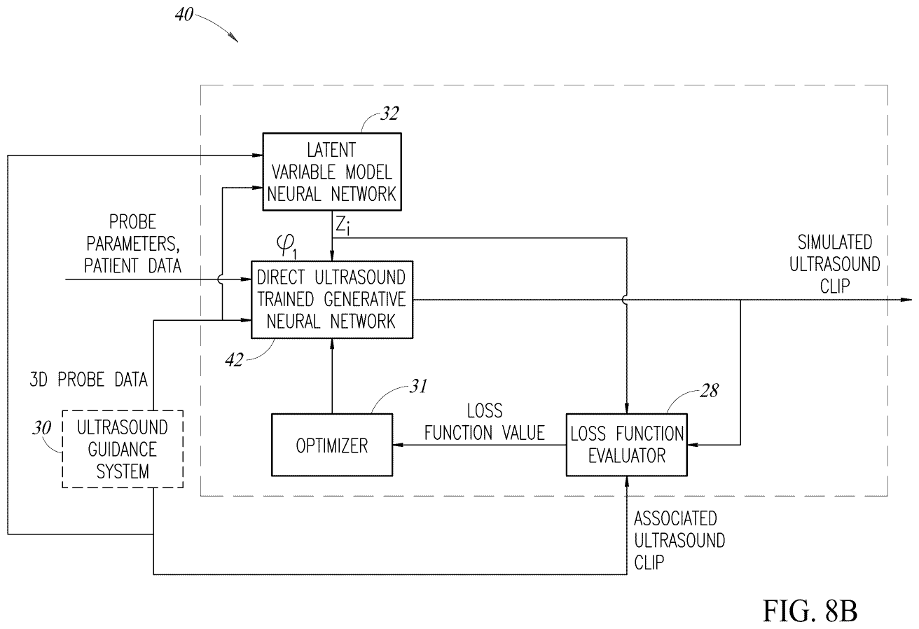

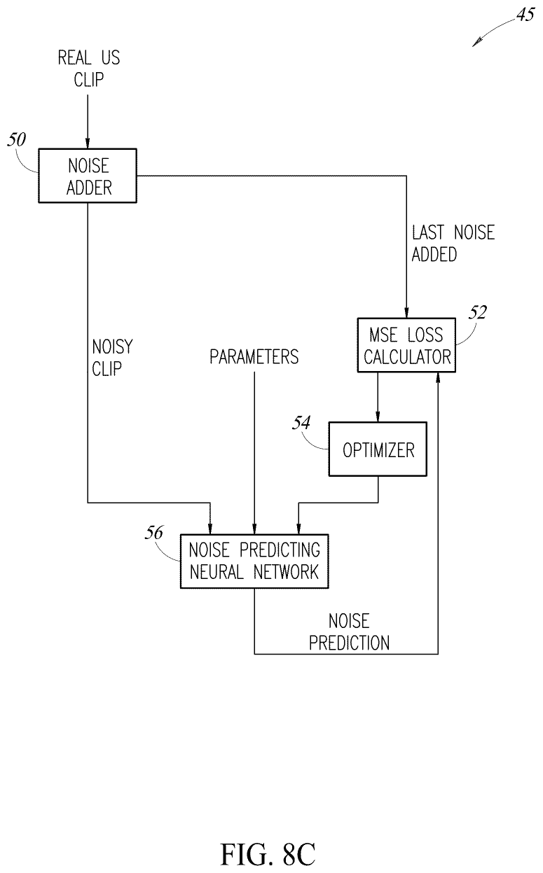

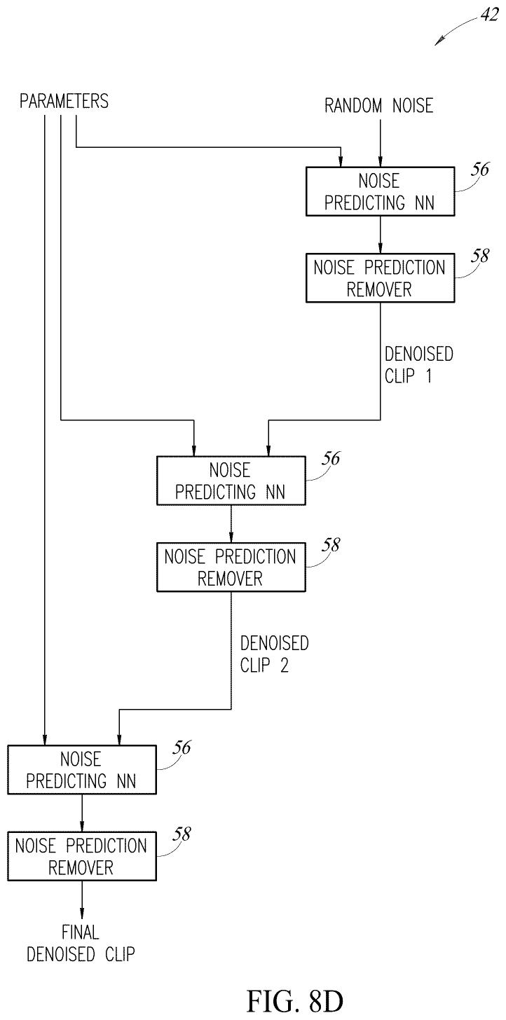

A second embodiment, shown in FIGS. 8A and 8B to which reference is now made, shows a simulator 40/40′ which may be implemented with a single, direct, ultrasound trained, generative neural network 42, trained on 2D ultrasound images or clips and associated data, as in the previous embodiment. FIG. 8B shows the embodiment with latent variable model neural network 32 while FIG. 8A shows the embodiment without it.

Simulator 40/40′ may be implemented as a diffusion model generative neural network which generates a trained neural network that takes an image of random noise and, through multiple ‘denoising’ steps, converts the image of random noise to a desired image, in response to the input data. In accordance with the present invention, direct ultrasound trained, generative neural network 42 may generate an ultrasound image, associated with the input probe parameters, patient parameters, and probe position and orientation, from an image of random noise.

To create neural network 42, it first needs to be trained. In accordance with a preferred embodiment of the present invention and as in the previous embodiment, the training dataset comprises pairs of ultrasound clips and their associated probe parameters and/or position and orientation and/or patient parameters.

FIG. 8C, to which reference is now made, illustrates the elements of a trainer 45, to train direct ultrasound generative neural network 42. The elements are a noise adder 50, a MSE (mean square error) loss calculator 52, an optimizer 54 and a noise predicting neural network 56. Each training sample may comprise a real 2D ultrasound clip, and its associated probe position and orientation, and any associated patient parameters, noise parameters, or operator parameters. These are shown in FIG. 8C generally as “parameters”. The training process of FIG. 8C may iterate multiple times in order to convert the input 2D ultrasound clip into a random image.

For each training sample it receives, noise adder 50 may add a random amount of noise or random distortions, to the ultrasound clip. Noise adder 50 may have a predefined procedure that adds a fixed amount of noise to each clip and may activate this procedure a random number Nᵢ of times in order to generate a random amount of noise to the clip. For the early iterations, noise adder 50 the fixed amount of noise may be small, such that the clip does not change much with the additional noise. For later iterations, when the clip images are very noisy already, the fixed amount of noise may be larger. Noise adder 50 may keep track of the amount of noise most recently added to the clip.

Noise predicting neural network 56, which may be implemented as a convolutional neural network, may receive both the noisy clip, and the general probe parameters associated with it, and may predict the noise that was added to the clip in the last iteration.

MSE loss calculator 52 may compare the prediction, generated by noise predicting neural network 56, to the last noise that was actually added to the clip, provided by noise adder 50, using an MSE loss. Optimizer 54 may then use the MSE loss to update the weights of noise predicting neural network 56. MSE loss calculator 52 and optimizer 54 may operate many times until noise predicting neural network 56 may generally correctly predict the noise that was added to the clip by noise adder 50 in each iteration.

The trainer 42 may repeat its operation many times (e.g. from 100-1000 timesor more), over different batches of training samples and different amounts of noise adding steps, until each clip has so much noise added to it that it is an entirely random clip. At this point, noise predicting neural network 56 may be trained and may be used in direct ultrasound generative neural network 42 to generate synthetic ultrasound clips for an input set of general probe parameters.

FIG. 8D, to which reference is now made, shows the elements of direct ultrasound generative neural network 42 using noise predicting neural network 56 and a noise prediction remover 58. Starting from a clip of totally random noise, noise predicting neural network 56 may predict a noise to remove, and noise prediction remover 58 may remove the predicted noise from the clip. This process may be repeated multiple times, for the same number of steps that were used by trainer 45. In this manner, the noise may be gradually removed from the initial random image, until a synthetic ultrasound clip is generated for the received general probe parameters.

Noise adder 50 may implement the following exemplary noise adding procedure:

xₜ₊₁=\sqrt{1−βₜ}·xₜ+\sqrt{βₜ}·∈

where xₜ₊₁ is the clip with noise added, xₜ is the clip before the noise is added, E is a random noise value generated from a standard normal distribution, and βₜ is a parameter that dictates how much of the random noise is added to the clip at iteration t.

Noise adder 50 may change βₜ over time, where an exemplary schedule for βₜ is:

$$\\ {β}_{t} = {β}_{start} \times {( \frac{{β}_{end}}{{β}_{start}} )}^{\frac{t}{T}}$$

Where β_{end} and βₛₜₐᵣₜ are predefined, fixed values and T is the maximum number of iterations that noise adder 50 may perform.

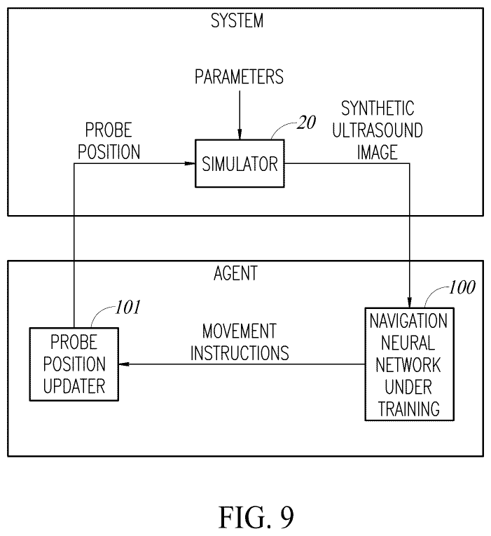

Any of the trained versions of simulators 20, 20′, 40 and 40′ may be utilized in a number of different applications, as shown in FIGS. 9, 10 and 11, to which reference is now made. The following description is written for simulator 20; it will be appreciated that any of the simulators described hereinabove may be used instead.

FIG. 9 illustrates a system producing synthetic data for machine learning systems. In this embodiment, one or more parameters, as well as the probe position and orientation, may be provided to simulator 20 which generates, therefrom, a synthetic ultrasound image of the body part associated with a position and orientation.

For example, one kind of machine learning system may be a classifier, which may attempt to classify aspects of the synthetic images or clips produced by simulator 20, a segmenter, which may segment an image into the objects therein and a regressor. For each of these, a user may convert the probe position and orientation and/or any of the other parameters into an annotation to provide the classification, segmentation or regression.

In an alternative embodiment and as shown in FIG. 9, one kind of machine learning system may be an ultrasound navigation neural network 100 under training. The navigation unit 100 may use the synthetic ultrasound image to determine training movement instructions to the probe, to achieve a desired view of the body part. Unit 100 may translate the movement instructions into the updated probe position, which may then be provided to simulator 20 to generate the next synthetic ultrasound image. Unit 100 may include a loss function and training unit to update its neural network based on the results.

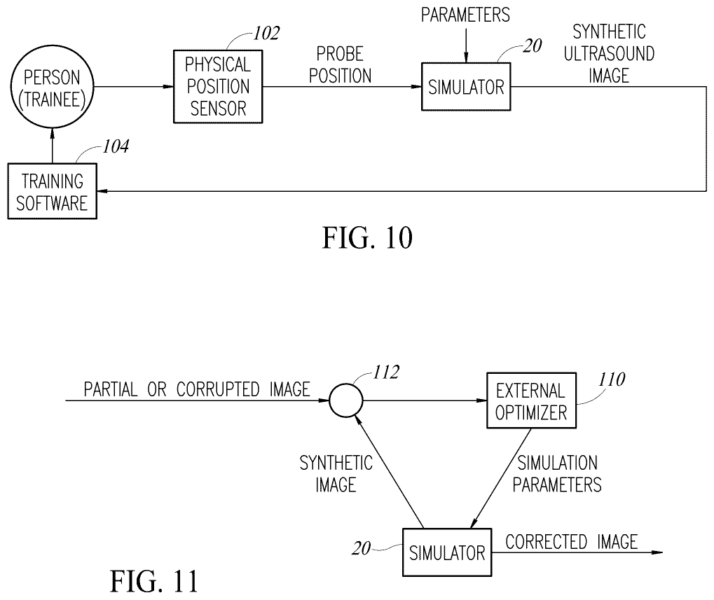

In FIG. 10, a physical device 102 with a position/orientation input sensor may be used together with simulator 20 to produce ultrasound images in real time that correspond to the sensor's position/orientation, as well as the patient parameters and/or the probe parameters. This system can be used as a training aid for sonographers, in conjunction with sonographer training software 104.

The trainee may move the probe whose position is provided to simulator 20 which may, in response, generate an ultrasound image of the body part associated with the position. The ultrasound image is then provided to the sonographer training software 104.

In accordance with a preferred embodiment of the present invention and as shown in FIG. 11, simulator 20 may be utilized to correct poor ultrasound images. In this embodiment, which comprises simulator 20 with an external optimizer 110, a partial or corrupted image may be provided to a comparator 112 which may determine a difference between the partial or corrupted image and a synthetic image generated by simulator 20. External optimizer 110 may update a set of simulation parameters for simulator 20 based on the difference produced by comparator 112. The simulation parameters may be any of the parameter discussed hereinabove, as well as the unknown probe position and orientation.

Simulator 20 may then generate an updated synthetic image for comparator 112 and the process may be repeated for a number of iterations, typically until the improvement in the synthetic image is small. At that point, simulator 20 may output the current synthetic image, which may be an improved version of the poor image.

The system of FIG. 11 may be utilized to provide image completion and/or image correction and/or image noise reduction and/or 3D reconstruction and/or new view synthesis, given sparse or corrupted ultrasound image data. The system of FIG. 11 may be used with any imaging data, such as TEE (Transesophageal Echocardiography), TTE (Transthoracic Echocardiography), ICE (Intracardiac Echocardiography), CT, MRI, Nuclear imaging, Xray, Fluroscopy.

Optimizer 110 may implement a gradient descent optimization on the simulation parameters. The simulation parameters may initially have random values. Optimizer 110 may change them to make the synthetic images closer to the partial images.

Unless specifically stated otherwise, as apparent from the preceding discussions, it is appreciated that, throughout the specification, discussions utilizing terms such as “processing,” “computing,” “calculating,” “determining,” or the like, refer to the action and/or processes of a general purpose computer of any type, such as a client/server system, mobile computing devices, smart appliances, cloud computing units or similar electronic computing devices that manipulate and/or transform data within the computing system's registers and/or memories into other data within the computing system's memories, registers or other such information storage, transmission or display devices.

Embodiments of the present invention may include apparatus for performing the operations herein. This apparatus may be specially constructed for the desired purposes, or it may comprise a computing device or system typically having at least one processor and at least one memory, selectively activated or reconfigured by a computer program stored in the computer. The resultant apparatus when instructed by software may turn the general purpose computer into inventive elements as discussed herein. The instructions may define the inventive device in operation with the computer platform for which it is desired. Such a computer program may be stored in a computer readable storage medium, such as, but not limited to, any type of disk, including optical disks, magnetic-optical disks, read-only memories (ROMs), volatile and non-volatile memories, random access memories (RAMs), electrically programmable read-only memories (EPROMs), electrically erasable and programmable read only memories (EEPROMs), magnetic or optical cards, Flash memory, disk-on-key or any other type of media suitable for storing electronic instructions and capable of being coupled to a computer system bus. The computer readable storage medium may also be implemented in cloud storage.

Some general purpose computers may comprise at least one communication element to enable communication with a data network and/or a mobile communications network.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the invention as described herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An ultrasound image simulator comprising:
a generative neural network to receive ultrasound probe position and orientation and to generate at least one simulated ultrasound image or clip of a body part of a subject,
said generative neural network being trained on a multiplicity of 2D ultrasound images or clips of said body part taken from a plurality of ultrasound probe positions and orientations.

2. The simulator of claim 1 wherein said multiplicity of 2D ultrasound images or clips and said plurality of ultrasound probe positions and orientations are generated by an ultrasound guidance system.

3. The simulator of claim 1 wherein said plurality of ultrasound probe positions and orientations are generated by a probe motion sensing system.

4. The simulator of claim 1 wherein said images or clips are associated with a multiplicity of parameters.

5. The simulator of claim 4 wherein said parameters comprise at least one of: parameters of an ultrasound probe, patient parameters, operator parameters and noise parameters.

6. The simulator of claim 5 wherein said at least one simulated ultrasound image or clip has an artificial or non-human element therein.

7. The simulator of claim 1 and wherein said generative neural network also comprises a latent variable provider.

8. The simulator of claim 7 and wherein said generative neural network is trained with a latent variable neural network receiving said multiplicity of 2D ultrasound images or clips and said plurality of ultrasound probe positions and orientations.

9. The simulator of claim 1 wherein said generative neural network comprises a diffusion model neural network.

10. The simulator of claim 1 wherein said generative neural network comprises:
a volumetric neural network to receive said ultrasound probe position and orientation and generating volume data of a body part for said ultrasound probe position and orientation;
a slicer to extract a slice of said volume associated with the position and orientation of said ultrasound probe; and
a rendering neural network to render said slice as a simulated ultrasound image of said body part.

11. The simulator of claim 10 and wherein both said volumetric neural network and said rendering neural network are trained with an empirical risk minimization training procedure and utilize the same loss function.

12. A system producing synthetic data for training an ultrasound based machine learning unit, the system comprising:
the simulator according to claim 4 to receive at least one of said multiplicity of parameters and to generate at least one said simulated ultrasound image or clip for said at least one parameter.

13. The system according to claim 12 and wherein said ultrasound based machine learning unit is one of: a classifier, a segmenter and a regressor.

14. The system according to claim 12 and wherein said ultrasound based machine learning unit comprises a navigation neural network under training to generate probe movement instructions for an ultrasound probe on a virtual subject and a probe position updater to convert said movement instructions to position and orientation of said ultrasound probe.

15. A system producing synthetic data for training a person to perform ultrasound scans of an artificial body with an ultrasound probe, the system comprising:
the simulator according to claim 4 to receive at least one of said multiplicity of parameters and to generate at least one said simulated ultrasound image or clip for said at least one parameter; and
sonographer training software to receive said at least one simulated ultrasound image or clip and to provide instructions to said person.

16. A system to improve a partial or corrupted image of a body part, the system comprising:
the simulator according to claim 4 to generate a simulated ultrasound image of said body part;
a comparator to determine a difference between said simulated ultrasound image with said partial or corrupted image; and
an optimizer to update said multiplicity of parameters and/or said ultrasound probe position and orientation to reduce said difference, thereby to produce an improved version of said partial or corrupted image of said body part.

17. The system according to claim 16 and wherein an output of said system is image completion and/or image correction and/or image noise reduction and/or 3D reconstruction and/or new view synthesis.

18. A method for generating an ultrasound image, the method comprising:
generating at least one simulated ultrasound image or clip of a body part of a subject with a generative neural network in response to ultrasound probe position and orientation,
said generative neural network being trained on a multiplicity of 2D ultrasound images or clips of said body part taken from a plurality of ultrasound probe positions and orientations.

19. The method of claim 18 and comprising generating said multiplicity of 2D ultrasound images or clips and said plurality of ultrasound probe positions and orientations by an ultrasound guidance system.

20. The method of claim 18 and comprising generating said plurality of ultrasound probe positions and orientations by a probe motion sensing system.

21. The method of claim 18 wherein said images or clips are associated with a multiplicity of parameters.

22. The method of claim 21 wherein said parameters comprise at least one of: parameters of an ultrasound probe, patient parameters, operator parameters and noise parameters.

23. The method of claim 22 wherein said at least one simulated ultrasound image or clip has an artificial or non-human element therein.

24. The method of claim 18 and also comprising providing latent variables to said generative neural network.

25. The method of claim 24 and comprising training said generative neural network with a latent variable neural network receiving said multiplicity of 2D ultrasound images or clips and said plurality of ultrasound probe positions and orientations.

26. The method of claim 18 wherein said generative neural network comprises a diffusion model neural network.

27. The method of claim 18 wherein said generating comprises:
generating volume data of a body part for said ultrasound probe position and orientation with a volumetric neural network in response to said ultrasound probe position and orientation;
extracting a slice of said volume associated with the position and orientation of said ultrasound probe; and
rendering said slice as a simulated ultrasound image of said body part with a rendering neural network.

28. The method of claim 27 and also comprising training both said volumetric neural network and said rendering neural network with an empirical risk minimization training procedure that utilizes the same loss function.

29. A method for producing synthetic data for training an ultrasound based machine learning unit, the method comprising:
receiving at least one of said multiplicity of parameters; and
generating at least one said simulated ultrasound image or clip for said at least one parameter with said generative neural network according to claim 21.

30. The method according to claim 29 and wherein said ultrasound based machine learning unit is one of: a classifier, a segmenter and a regressor.

31. The method according to claim 29 and wherein said ultrasound based machine learning unit comprises a navigation neural network under training which generates probe movement instructions for an ultrasound probe on a virtual subject and the method comprises converting said movement instructions to position and orientation of said ultrasound probe.

32. A method for producing synthetic data for training a person to perform ultrasound scans of an artificial body with an ultrasound probe, the method comprising:
receiving at least one of said multiplicity of parameters;
generating at least one said simulated ultrasound image or clip for said at least one parameter with said generative neural network according to claim 21; and
a sonographer training software providing instructions to said person.

33. A method to improve a partial or corrupted image of a body part, the method comprising:
generating at least one said simulated ultrasound image or clip with said generative neural network according to claim 21;
determining a difference between said simulated ultrasound image with said partial or corrupted image; and
updating said multiplicity of parameters and/or said ultrasound probe position and orientation to reduce said difference, thereby to produce an improved version of said partial or corrupted image of said body part.
